(19) United States
(12) Patent Application Publication
(10) Pub. No.: US 2016/0128158 A1
(43) Pub. Date: May 5, 2016

(54) LED ENVIRONMENT ENGINE

(71) Applicant: Samsung Electronics Co., Ltd., Gyeonggi-Do (KR)

(72) Inventor: Frank HARDER, San Jose, CA (US)

(21) Appl. No.: 14/919,560

(22) Filed: Oct. 21, 2015

Related U.S. Application Data
(60) Provisional application No. 62/075,758, filed on Nov. 5, 2014.

(51) Int. Cl. H05B 33/08; A61B 5/0205; H05B 37/02

(52) U.S. Cl. CPC H05B 33/0872; H05B 37/0272; H05B 37/0227; H05B 37/0236; H05B 37/0218; A61B 5/02055

(57) ABSTRACT

An environment engine with LED lighting is disclosed herein. According to one embodiment, a lighting system is disclosed having a sensor that detects a physical condition of a user. The physical condition includes one or more of a heart rate, a body temperature, and a rate of motion. The lighting system further includes a processor that receives an identification of the user via a radio module that communicates with a mobile device. The processor controls a lighting module based on the physical conditions detected by the sensor and the identification of the user.

### CROSS-REFERENCE TO RELATED APPLICATIONS

The present application claims priority to provisional application Ser. No. 62/075,758, filed on Nov. 5, 2014, entitled “LED ENVIRONMENT ENGINE” naming the same inventors as in the present application. The contents of the above referenced provisional application are incorporated by reference, the same as if fully set forth herein.

### FIELD

The present disclosure generally relates to the field of light-emitting diode (LED) lighting systems. More specifically, the present disclosure is related to environment engines having sensors, power management, processing power, analytics, algorithms, and software capabilities in combination with LED Illumination.

### BACKGROUND

Incandescent and florescent lighting have long been used to light our homes, public spaces, and places of business. Recently, LED lighting systems have gained popularity by offering several key advantages. For example, LED lighting systems offer a long lifetime of use, typically up to 11 years of continuous operation, and are approximately 70% more energy efficient compared to traditional lighting sources. Due to energy shortages as well increased overall energy pricing, a widespread adoption of LED lighting is taking place alongside an increased desire for more advanced features in controls and connectivity to further improve the quality and efficiency of these systems. For example, lights are frequently left on when a room is unoccupied, causing an unnecessary consumption of power and a decrease in the useful life of the LED.

At the same time, communication and processing components, such as the kind typically found in cellular phones and tablets, for example, are becoming more common and less expensive. By integrating these processors, sensors and communication components into lighting systems, advanced features may be used to enhance environmental conditions and make these lighting systems more ecologically friendly and useful in combination with further applications in controls and automation.

### SUMMARY

According to one embodiment, a lighting system is disclosed having a sensor that detects a physical condition of a user. The physical condition includes one or more of a heart rate, a body temperature, and a rate of motion. The lighting system further includes a processor that receives an identification of the user via a radio module that communicates with a mobile device, where the processor controls a lighting module based on the physical conditions detected by the sensor and the identification of the user.

According to a second embodiment, a method is disclosed that includes detecting a physical condition of a user using a sensor, where the physical condition includes one or more of a heart rate, a body temperature, and a rate of motion, receiving an identification of the user via a radio module that communicates with a mobile device, and controlling a lighting module based on the physical conditions detected by the sensor and the identification of the user.

### BRIEF DESCRIPTION OF THE DRAWINGS

The accompanying drawings, which are incorporated in and form a part of this specification, illustrate embodiments of the present disclosure and, together with the description, serve to explain the principles of the present disclosure:

FIG. 1 is a diagram of an exemplary environment engine having external LED lighting, according to one embodiment.

FIG. 2 is a diagram of an exemplary environment engine having integrated LED lighting, according to one embodiment.

FIG. 3 is a diagram depicting an exemplary environment engine installed in a living room of a home or apartment and connected to external devices, according to one embodiment.

FIG. 4 is a diagram depicting an exemplary environment engine installed in a municipal parking lot and connected to an external computer system, according to one embodiment.

FIG. 5 is a flowchart depicting an exemplary environment engine installed at a municipal street corner, according to one embodiment.

FIG. 6 is a flowchart depicting an exemplary method for controlling a lighting module, according to one embodiment.

### DETAILED DESCRIPTION

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

Portions of the detailed description that follows are presented and discussed in terms of a method. Embodiments are well suited to performing various other steps or variations of the steps recited in the flowchart of the figures herein, and in a sequence other than that depicted and described herein.

Some portions of the detailed description are presented in terms of procedures, steps, logic blocks, processing, and other symbolic representations of operations on data bits that can be performed on computer memory. These descriptions and representations are the means used by those skilled in the data processing arts to most effectively convey the substance of their work to others skilled in the art. A procedure, computer-executed step, logic block, process, etc., is here, and generally, conceived to be a self-consistent sequence of steps or instructions leading to a desired result. The steps are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout, discussions utilizing terms such as “accessing,” “writing,” “including,” “storing,” “transmitting,” “traversing,” “associating,” “identifying” or the like, refer to the action and processes of a computer or other electronic computing device that manipulates and transforms data represented as physical (electronic) quantities within the system's registers and memories into other data similarly represented as physical quantities within the system memories or registers or other such information storage, transmission or display devices.

### LED Environment Engine

An environment engine having radio and sensor subsystems is disclosed herein. According to some embodiments, an LED lighting component is housed within the environment engine. According to other embodiments, LED lighting may be provided by an external array or device coupled to the environment engine. Wired communications may be provided by Ethernet (e.g., Power over Ethernet (PoE)), USB, or Power Line Communication. The LED environment engine includes a processor, memory, a sensor subsystem, and a power management unit. According to some embodiments, the power management unit includes an AC/DC conversation unit with connectivity and a firmware layer. Wireless connectivity may be provided by one or more onboard transceivers (e.g., Long-Term Evolution (LTE), code division multiple access (CDMA), Global System for Mobile Communications (GSM), high speed packet access (HSPA), WiFi, Bluetooth, ZIGBEE®, Z-WAVE™, near field communications (NFC), and infrared (IR)) that may be coupled to one or more antennae to increase reception. A programmable logic controller (PLC) may also be included and may be used for configuring the environment engine using an external device. Furthermore, the LED environment engine includes a processor, memory, an environmental sensor subsystem, and a power management unit. The wireless and/or wired communications received by the environment engine and sensor data received by the environmental sensor subsystem are used by the processor and/or main memory to provide enhanced environmental services (e.g., light-as-a-service, HVAC)

According to one embodiment, a lighting system is disclosed having a processor and memory, a sensor subsystem including a camera, and a radio subsystem including a radio for providing communication with an external device. The processor controls an LED or an external device based on environmental conditions detected by the sensor subsystem and control signals received by the radio subsystem from an external device.

According to another embodiment, a method of controlling environmental conditions is disclosed. The method includes receiving sensor data from a sensor subsystem, the sensor subsystem having a camera, receiving a control signal from an external device using a radio subsystem, the radio subsystem having a radio for providing communication with an external device, and controlling an LED or external device using a processor based on environmental conditions detected by the sensor subsystem and control signals received by the radio subsystem from an external device to affect one or more environmental conditions.

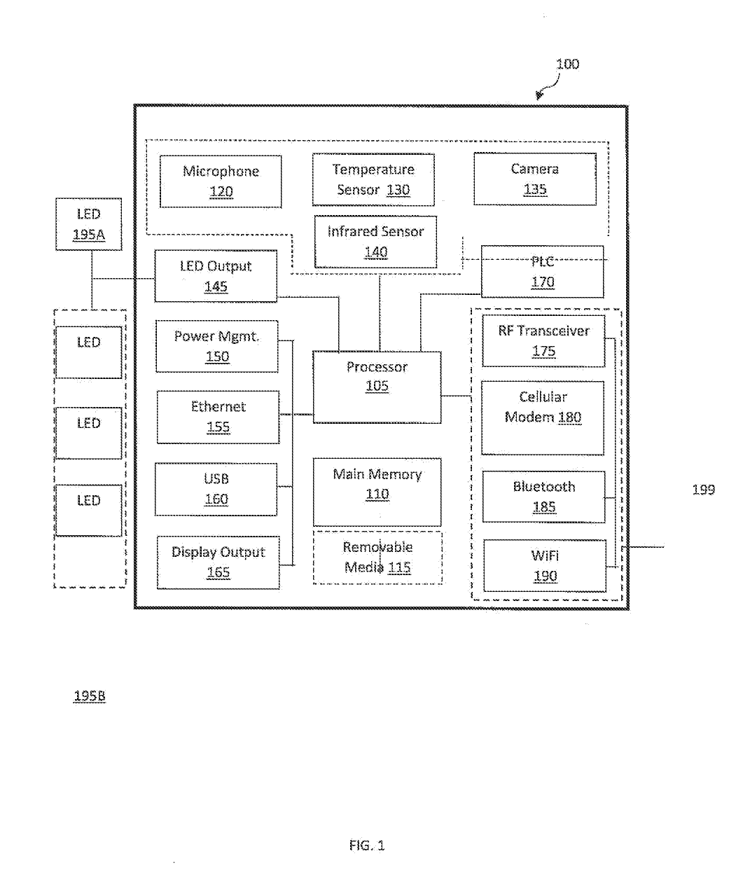

With regard to FIG. 1, environment engine 100 is depicted according to embodiments of the present disclosure. Processor 105 is coupled to main memory 110. Main memory 110 may include, but is not limited to, RAM, ROM, EEPROM, flash memory or other memory technology, or any other medium which may be used to store the desired information and which may be accessed by a computing device. Processor 105 may be a general purpose processor or a specialized processor and may have one or more cores. According to some embodiments, removable media 115 may include an SD or MMC card, for example, and may be accessed by processor 105 to store and/or retrieve data using removable media. Processor 105 is also coupled to an environmental sensor subsystem, a radio subsystem, a power management unit 150, an Ethernet module 155, a USB module 160, a LED output module 145, and a display output module 165. Programmable logic controller 170 may be used for configuring the environment engine using an external device.

The radio subsystem includes at least one of a radio frequency (RF) transceiver 175, a cellular modem 180, a Bluetooth module 185, and a WiFi module 190. The radio subsystem and/or its components are coupled to processor 105. According to some embodiments, the radio subsystem and/or its components may be coupled to an internal or external antenna 199. The antenna may be active (e.g., powered by an external source) or passive and enhances the ability of the radio subsystem to send and receive data.

An environmental sensor subsystem coupled to processor 105 may include a microphone 120, a temperature sensor 130, an infrared sensor 140, and a camera 135. Processor 105 is coupled to power management unit 150 for providing power (e.g., an AC or DC signal) to environment engine 100. Power management unit 150 may be powered by an external source or an internal source, such as a rechargeable or replaceable battery. Environment engine 100 may be communicatively coupled to an external device (e.g., a computer, a control unit) using Ethernet module 155, USB module 160 or a wireless connection using components of the radio subsystem (e.g., RF transceiver 175, cellular modem 180, Bluetooth module 185, and WiFi module 190). The external device may receive environmental information from environment engine 100 and send control data to environment engine 100 to activate and/or control a feature of environment engine 100. According to some embodiments, environment engine 100 may output display information to a display device (e.g., a monitor, a TV) using display output module 165. Display output module 165 may support a variety of connections (e.g., HDMI, composite, DVI). According to some embodiments, environment engine 100 may interface with a heating, ventilation, and air conditioning system (HVAC) to activate and/or control one or more features of the HVAC system using USB module 160, Ethernet module 155, or a wireless interface of the radio subsystem (e.g., RF transceiver 175, cellular modem 180, Bluetooth 185, and WiFi 190).

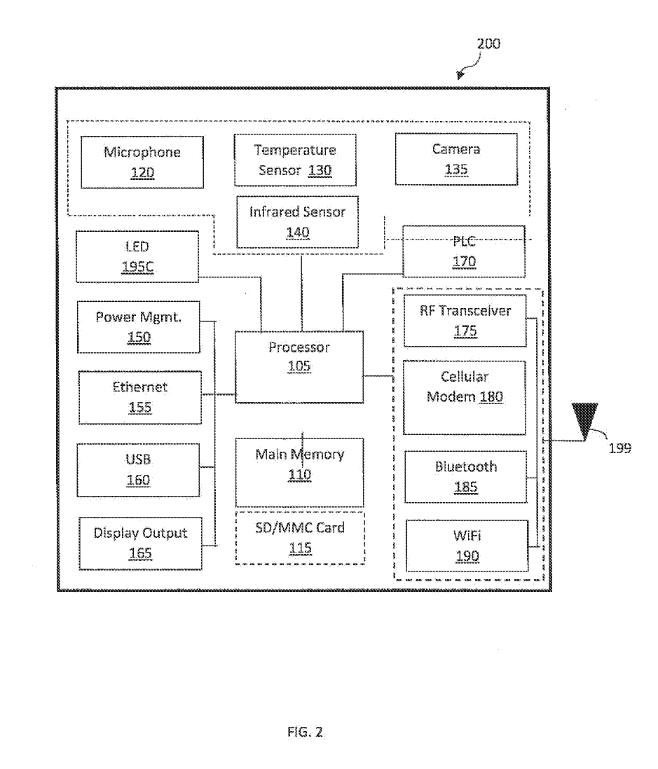

With regard still to FIG. 1, LED output module 145 is coupled to processor 105 and one or more LED lighting units or arrays (e.g., LED unit 195A and LED array 195B). LED lighting unit 195A may include a single diode controlled by LED output module 145 to controllably emit light. LED array 195B may include a series of diodes controlled by LED output module 145 to controllably emit light. As depicted in FIG. 2, an integrated LED unit 195C may be housed within environment engine 200 and coupled to processor 105 to controllably emit light, according to one embodiment.

Sensor data received by the environmental sensor subsystem may be communicated to the processor and/or memory. Environment engine 100 may use data received from sensors (e.g., microphone 120, temperature sensor 130, infrared sensor 140, and camera 135) to activate or configure lighting and environmental services. Lighting services may include turning an LED on or off, diming or increasing light emitted from an LED, or changing an output color of an LED. According to some embodiments, the sensor subsystem may include a microphone that detects sounds and/or voice commands. For example, the voice command “lights on” may be detected by microphone 120 and cause processor 105 to send a control signal to turn on an LED (e.g., LED unit 195A, LED array 195B, or integrated LED unit 195C). As another example, a noise level above a predetermined threshold may be detected by microphone 120 and indicate the presence of one or more individuals, causing processor 105 to send a control signal to activate a feature of environment engine 100 (e.g., turn on an LED, adjust HVAC settings, and send a signal using the radio subsystem).

According to some embodiments, the sensor subsystem may include a camera that detects motion and/or lighting conditions. For example, camera 135 may detect motion indicating the presence of an individual causing processor 105 to send a control signal to activate a feature of environment engine 100 (e.g., turn an LED on or off, adjust HVAC settings, and send a signal using the radio subsystem). As another example, camera 135 may detect a brightness level of the environment above or below a predetermined threshold and cause processor 105 to send a control signal to activate a feature of environment engine 100 (e.g., turn an LED on or off, adjust HVAC settings, and send a signal using the radio subsystem).

According to some embodiments, the sensor subsystem may include a temperature sensor that detects an ambient temperature near environment engine 100. For example, temperature sensor 130 may detect a temperature above or below a predetermined threshold, causing processor 105 to send a control signal to activate a or adjust an environmental service or condition (e.g., turn an LED on or off, activate or adjust an HVAC setting, and send a signal using the radio subsystem).

According to some embodiments, the sensor subsystem may include an infrared sensor that detects infrared light around environment engine 100. For example, infrared sensor 140 may detect infrared heat or light indicating the presence of an individual and cause processor 105 to activate a feature of environment engine 100 (e.g., turn an LED on or off, adjust HVAC settings, and send a signal using the radio subsystem). As another example, infrared sensor 140 may detect a body heat of an individual above or below a predetermined threshold and cause processor 105 to activate a feature of environment engine 100 (e.g., turn an LED on or off, adjust HVAC settings, and send a signal using the radio subsystem).

Control data received by the radio subsystem may be used to control or monitor conditions of environment engine 100. For example, a client device may connect to environment engine 100 using a wired connection (e.g., USB, Ethernet) or a wireless connection using a component of the radio subsystem (e.g., RF transceiver 175, cellular modem 180, Bluetooth module 185, and WiFi module 190). The client device may use an application or web portal running on the client device to interface with and control environment engine 100. According to one embodiment, a client device may send identification and/or security information to environment engine 100 to establish a secure connection and/or identify a user of the client device. Furthermore, the radio subsystem is operable to establish and/or join an ad hoc or mesh network including of other environment engines and/or client devices.

According to some embodiments, one or more component of the radio subsystem (e.g., RF transceiver 175, cellular modem 180, Bluetooth module 185, and WiFi module 190) may be used to connect environment engine 100 to other environment engine or similar devices. For example, the environment engine 100 may wirelessly connect to an Internet-of-things (IoT) platform to send data or receive data and/or control signals from an external device. Furthermore, the radio subsystem may transmit data (e.g., environmental data) to one or more client devices. The transmitted data may relate to information captured by an environmental sensor subsystem, such as room temperature, number or identity of room occupants, HVAC status (e.g., heating, cooling, circulation, heating schedule, and cooling schedule), motion detection information, visual information captured by a camera or infrared sensor, or information gathered from connected client devices, for example. Information gathered form client devices may include, but not limited to, an identity of an individual (e.g., a name, a social security number, an employee ID, and a username), location information, payment information, phone number, browser history, device information (e.g., a hardware specification, a model number), a service provider, a contact list, and social media data.

The features disclosed herein enable LED lighting with energy savings beyond 70%, and in some cases roughly 90 to 95% dependent on the application. According to some embodiments, the LED environment engine is further operable to provide smart metering of power consumption using the senor subsystem and/or the radio subsystem. Furthermore, embodiments of the LED environment engine discussed herein are operable to provide smart and/or real time flicker-free dimming. According to some embodiments, overall quality of light is improved using daylight quality lighting or Correlated Color Temperature (CCT) lighting. Further embodiments provide color changing LED lighting and/or changeable color rendering index (CRI) lighting. According to one embodiment, the present system provides an improved lighting spectrum and/or a circadian rhythm color pattern enabled by one or more sensors of a sensor subsystem in combination with one or more lighting algorithms to provide environmental enhancement effects. Examples of environmental enhancement effects provided by an improved lighting spectrum and/or a circadian rhythm color pattern produced by the LED environment engine include enhanced workplace productivity, enhanced learning cycles in schools, shortened recovery times in hospitals, and improved quality of life in elderly care facilities. Environmental enhancement effects provided by the LED environment engine may alter the circadian clock of patients to various effects. In one example, blue light content is provided to a patient to improve the quality and/or quantity of the patient's daytime activity. In another example, red and/or amber light content is provided during evening times to improve a patient's sleeping habits, health and wellness.

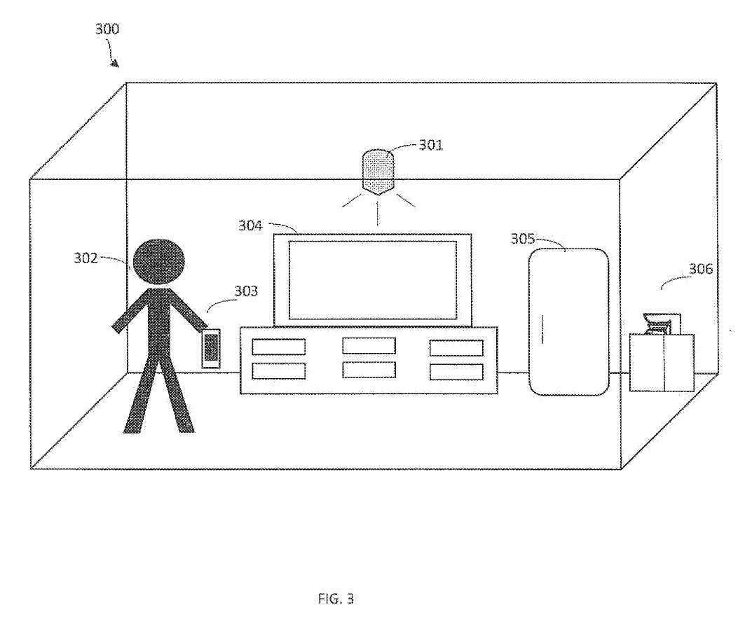

With regard to FIG. 3, an exemplary environment engine 301 includes an integrated LED unit installed in a living room of a home or apartment 300, according to one embodiment. Environment engine 301 connects to other devices in the home and may act as a hub for controlling lighting and various other appliances (e.g., a smart home appliance). The environment engine may attempt to identify occupants of the room using the sensor subsystem or the radio subsystem, or a combination of the two. The environment engine may include a processor that receives an identification of an individual via a radio module that communicates with the individual's mobile device, for example, via an application on the individual's mobile device.

For example, the environment engine may detect the presence of an individual in the room (e.g., occupant 302) using one or more components of the sensor subsystem (e.g., camera 135, microphone 120, and infrared sensor 140) and attempt to identify the individual, for example, by connecting the individual's smartphone (e.g., smartphone 303) using Bluetooth or WiFi. The individual may automatically be identified using a key or signature, or the individual may identify themselves using an application or web portal running on their smartphone. If an unrecognized or unauthorized user is detected, the system may trigger an alarm or other security features (e.g., turning on an LED, capturing video and/or audio). The environment engine may also monitor the health and wellbeing of individuals (e.g., occupant 302), for example, using the sensor subsystem to detect a physical condition of a user such as an abnormal condition (e.g., heart rate, body temperature, a rate of motion, and a lack of motion). The processor of the environment engine may control a lighting module based on the physical conditions detected by the sensor and the identification of the individual. The environment engine may also connect to other devices in the home using an IoT platform. For example, the environment engine may detect the presence of an individual and cause television 304, refrigerator 305, or coffee maker 306 to turn on and/or activate certain features over a wired (e.g., USB and Ethernet) or wireless (e.g., RF, cellular, Bluetooth, and WiFi) connection.

In another illustrative example, a plurality of exemplary environment engines connected to an LED array (e.g., environment engine 100) is installed in a place of business, such as an office building or retail store. Each room or office in the building may have a dedicated environment engine, and all of the Environment engines may be connected using a wired (e.g., USB and Ethernet) or wireless (e.g., RF, cellular, Bluetooth, and WiFi) interface. In one example, an environment engine acts as a hub, where other environment engines connect to the hub and send and receive data and/or control signals to and from the hub. In this way, the environment engine is expandable as new environment engines or lighting systems may be added and connected to the hub as needed. The sensor subsystem of the environment engines in each room may be used to determine when the room is occupied. This allows the LED lighting to be activated only when necessary to save lighting costs and extend the life of the lighting systems. Furthermore, the environment engine may connect to client devices (e.g., smartphones, tablets, and laptops of customers and employees) using wired or wireless technology. The environment engine may store data received from client devices and/or perform analytics on the received data. Furthermore, the environment engine may transfer the data to a local or remote device for storage and/or analysis. This data may provide valuable insight regarding demographics and interests of customers that have entered a retail store, for example.

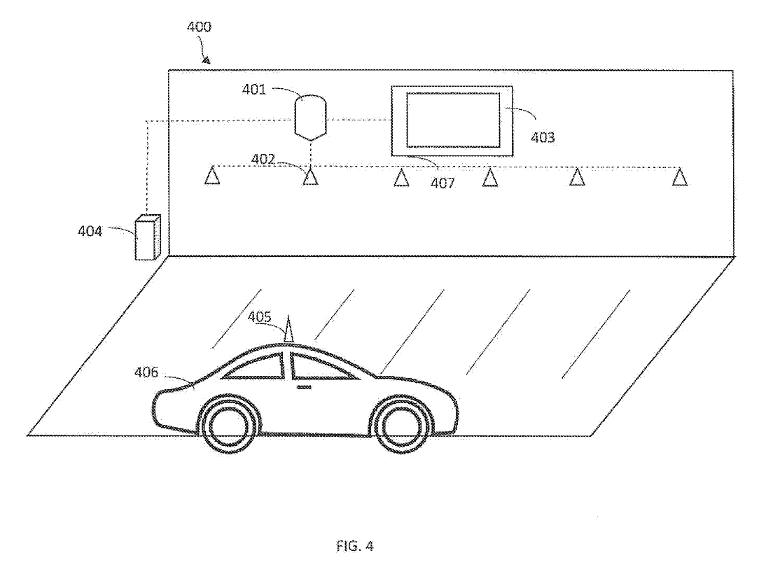

With regard to FIG. 4, an environment engine 401 is connected to an array of LEDs 402 and installed in a parking lot 400, according to one embodiment. Environment engine 401 is connected to an external display device 403 using display output 165 for displaying information. In this example, environment engine 401 is connected to an external computer system 404 using an Ethernet interface. External computer system 404 sends data to the environment engine including, but not limited to weather, traffic, public transportation, and parking data. The data sent from external computer system 404 is received by the environment engine and displayed on display device 403 and/or sent to client devices (e.g., communications system 405 of vehicle 406, a smartphone, and a laptop) connected to environment engine 401.

The environment engine provides lighting services using LED array 402. For example, when a senor detects that a brightness level around environment engine 401 is below a predetermined threshold, environment engine 401 may activate the LEDs of LED array 402. As another example, a sensor of environment engine 401 may determine that a vehicle (e.g., vehicle 406) has parked in a parking space proximate to LED unit 407. To indicate that the parking space is occupied, a processor of environment engine 401 causes LED unit 407 to emit red light. At a later time, the parking space becomes unoccupied and a processor of environment engine 401 causes LED unit 407 to emit green light indicating that the parking space is unoccupied. environment engine 401 may also provide security features to increase the safety of the parking lot by providing surveillance similar to a closed circuit television (CCTV) system using a camera (e.g., camera 135) and sending captured images to a client device or connected computer system 404 for monitoring or storage.

According to one embodiment, the present system provides smart parking and/or traffic applications using a combination of software (e.g., an application), sensors (e.g., an HD video camera), and one or more communications components of a radio subsystem. In one example, a traffic management and security solutions application may be executed by a processor to receive and/or store traffic, public transport and/or pedestrian safety information. Location tracking and other security features may be provided in private and public spaces (e.g., parking structures, airports, schools, geo fencing, and homeland security) using one or more LED lighting engines. For example, a series of LED lighting/environment engines may interoperate to track a vehicle using one or more cameras to detect a license plate number of the vehicle, as well as the vehicle's direction of movement, speed, and/or current location may be provided. The present environment engine may provide other security features including emergency escape guidance in case of a fire and other environmental catastrophe using one or more sensors of the sensor subsystem (e.g., a camera, a temperature sensor, and an infrared sensor). According to one embodiment, the radio subsystem is operable to send data to and receive data from a machine-to-machine (M2M) communications system. The M2M communications system enables features related to the operation of autonomous cars. The M2M communications system is further operable to provide traffic classification and security information to the LED environment engine.

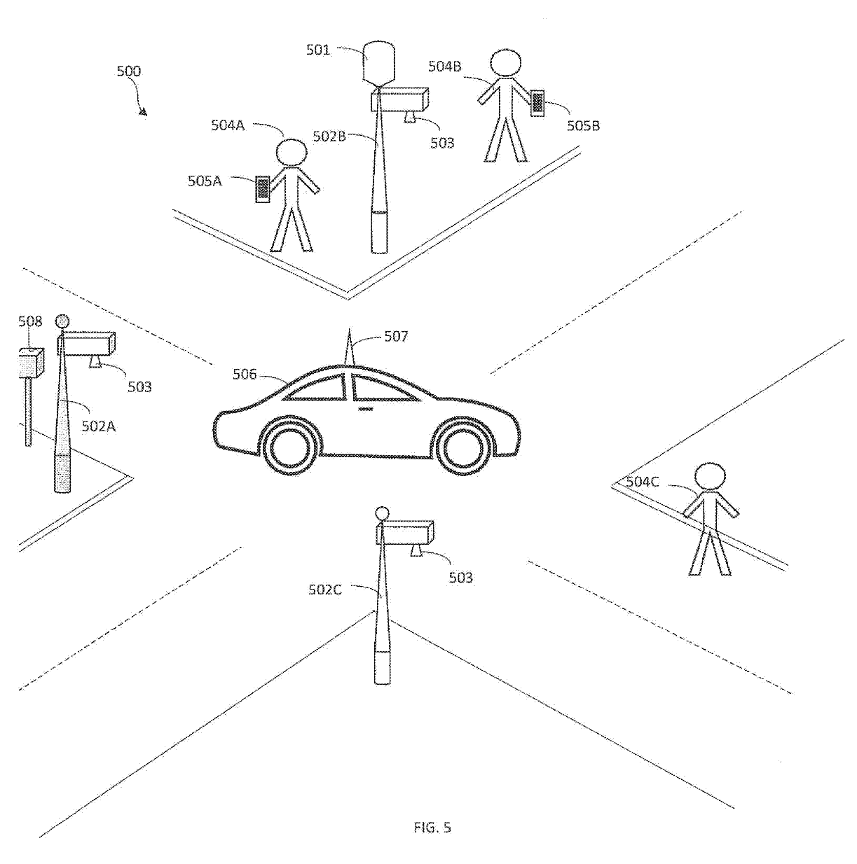

With regard to FIG. 5, an LED environment engine 501 is installed at a street corner 500, according to one embodiment. LED environment engine 501 is communicatively coupled to lighting structures 502A, 502B, and 502C having LED lighting (e.g., an LED unit 503) installed at the corners of intersection 500. As depicted, LED environment engine 501 is installed on top of lighting structure 502B, although other installation locations are possible (e.g., a street side utility box, underground, and inside a building structure). Using wireless communications (e.g., Wifi, Bluetooth, and RF) provided by a radio subsystem, LED environment engine 501 communicates with external devices such as communication system 507 of vehicle 506 and smartphones 505A and 505B. Using data received from external devices and/or components of a sensor subsystem (e.g., a camera, a microphone, and an Infrared sensor), the LED environment engine is operable to control aspects of the environment, such as lighting and safety.

In one example, a camera of LED environment engine 501 may detect a pedestrian 504A waiting to cross a street. By connecting to the pedestrian's smartphone 505A, a processor of LED environment engine 501 determines that pedestrian 504A intends to walk in the direction of lighting structure 502A. In response, LED environment engine 501 activates LED unit 503 of lighting structure 502A so the pedestrian may safely cross the well-lit street. Pedestrian 504C does not have a smartphone, however, a sensor subsystem of LED environment engine 501 visually identifies that pedestrian 504C has stepped off of the sidewalk and is crossing a street in the direction of lighting structure 502C. In response, LED environment engine 501 activates LED unit 503 of lighting structure 502C so the pedestrian may safely cross the well-lit street. Communication system 507 of vehicle 506 also connects to LED environment engine 501 using wireless communications technology. Communication system 507 sends traffic and weather information to a display device installed in vehicle 506 using communication system 507. Pedestrian 504B wirelessly connects to LED environment engine 501 using a wireless connection (e.g., cellular, WiFi, and Bluetooth) of smartphone 505B. LED environment engine 501 sends data to smartphone 505B indicating that crosswalk signal 508 will change to ‘WALK’ in 36 seconds. In the case of emergency, a pedestrian (e.g., pedestrian 504B) may use a connected smartphone (e.g., smartphone 505B) to send a distress signal to LED environment engine 501. In response, LED environment engine 501 alerts emergency services and/or stores a video and/or audio recording of intersection 500. According to some embodiments, LED environment engine 501 logs information regarding connected devices (e.g., smartphones 505A and 505B) and/or users (e.g., pedestrians 505A and 505B) at or near the intersection.

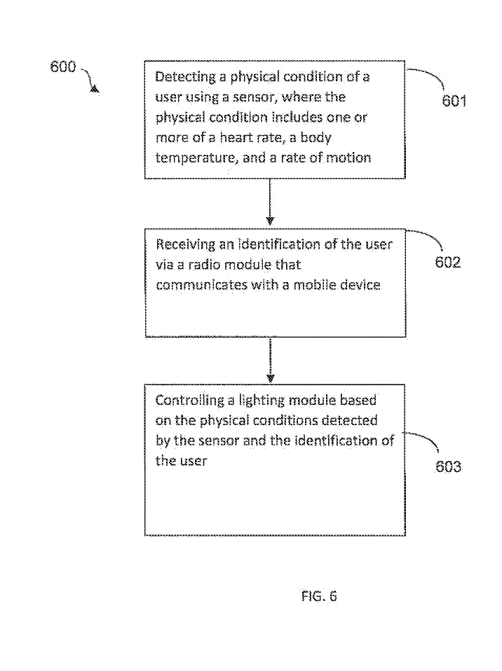

With regard to FIG. 6, flow chart 600 depicts a method of controlling a lighting module, according to one embodiment. The method includes detecting a physical condition of a user using a sensor at step 601. The physical condition may include a heart rate, a body temperature, and/or a rate of motion. An identification of the user is received via a radio module that communicates with a mobile device at step 602. At step 603, a lighting module is controlled based on the physical conditions detected by the sensor and the identification of the user.

Embodiments of the present disclosure are thus described. While the present disclosure has been described in particular embodiments, it should be appreciated that the present disclosure should not be construed as limited by such embodiments, but rather construed according to the following claims.

What is claimed is:

1. A system, comprising:
a sensor that detects a physical condition of a user, wherein the physical condition includes one or more of a heart rate, a body temperature, and a rate of motion; and
a processor that receives an identification of the user via a radio module that communicates with a mobile device, wherein the processor controls a lighting module based on the physical conditions detected by the sensor and the identification of the user.

2. The system of claim 1, wherein the sensor comprises at least one of a microphone, a temperature sensor, a camera, and an infrared sensor.

3. The system of claim 1, wherein the radio module comprises at least one of an RF transceiver, a cellular modem, a Bluetooth transceiver and a WiFi transceiver.

4. The system of claim 1, wherein the processor is operable to transmit information received by the radio module to an external device for storage.

5. The system of claim 1, wherein the lighting module comprises at least one LED controllable by the processor to change an output color of the LED.

6. The system of claim 5, wherein the lighting module comprises color rendering index lighting.

7. The system of claim 1, wherein the lighting module comprises an array of light sources coupled to an LED output module that controllably emits light.

8. A method, comprising:
detecting a physical condition of a user using a sensor, wherein the physical condition includes one or more of a heart rate, a body temperature, and a rate of motion;
receiving an identification of the user via a radio module that communicates with a mobile device; and
controlling a lighting module based on the physical conditions detected by the sensor and the identification of the user.

9. The method of claim 8, wherein the sensor further comprises at least one of a microphone, a temperature sensor, a camera, and an infrared sensor.

10. The method of claim 8, wherein the radio module comprises at least one of an RF transceiver, a cellular modem, a Bluetooth transceiver and a WiFi transceiver.

11. The method of claim 8, wherein the lighting module is controlled using an Internet-of-things platform.

12. The method of claim 8, wherein the lighting module is controlled using at least one of a smartphone application and a web portal.

13. The method of claim 8, further comprising transmitting information received by the radio module to an external device for storage

14. The method of claim 8, wherein the lighting module comprises at least one LED having an adjustable output color.

15. The method of claim 14, wherein the lighting module comprises color rendering index lighting.

16. The method of claim 8, wherein the lighting module comprises an array of light sources coupled to an LED output module that controllably emits light.

17. A non-transitory computer readable medium having stored thereon computer-readable instructions, and a processor coupled to the non-transitory computer readable medium, wherein the processor executes the computer-readable instructions to:
detect a physical condition of a user using a sensor, wherein the physical condition includes one or more of a heart rate, a body temperature, and a rate of motion;
receive an identification of the user via a radio module that communicates with a mobile device; and
control a lighting module based on the physical conditions detected by the sensor and the identification of the user.

18. The non-transitory computer readable medium of claim 17, wherein the lighting module is controlled using an Internet-of-things platform.

19. The non-transitory computer readable medium of claim 17, wherein the lighting module comprises at least one LED having an adjustable output color.

20. The non-transitory computer readable medium of claim 19, wherein the lighting module comprises color rendering index lighting.
